(19) United States
(12) Patent Application Publication
(10) Pub. No.: US 2009/0107217 A1
(43) Pub. Date: Apr. 30, 2009

(54) METHOD OF DETECTING METAL IONS BY MELANIN-SENSITIZED PIEZOELECTRIC SENSOR

(75) Inventor: Gue-Wha Huang, Jhunan Township (TW)

Correspondence Address: OCCHIUTI ROHLICEK & TSAO, LLP, 10 FAWCETT STREET, CAMBRIDGE, MA, 02138, US

(73) Assignee: Bureau of Animal and Plant Health Inspection and Quarantine, Council of Agriculture, Taipei (TW)

(21) Appl. No.: 12/110,872

(22) Filed: Apr. 28, 2008

(30) Foreign Application Priority Data
Oct. 29, 2007 (TW) 096140631

(51) Int. Cl. G01N 33/20; G01N 29/00

(52) U.S. Cl. 73/61.42; 73/61.79

(57) ABSTRACT

The present invention provides a metal-ion sensor characterized by coating the gold electrode of a quartz crystal microbalance (QCM) with melanin, whereby the sensor has an enhanced sensitivity to metal ions.

### CROSS-REFERENCE TO RELATED APPLICATIONS

This application claims the benefit of Taiwan Application No. 096140631, filed on Oct. 29, 2007. The contents of which is hereby incorporated by reference in its entirety.

### TECHNICAL FIELD

This invention relates to a melanin-sensitized piezoelectric sensor.

### BACKGROUND

A quartz crystal microbalance (QCM) is a mass-detection device that operates based on the piezoelectric properties of quartz crystals. The QCM is simple in structure, cost-effective, real-time responsive, highly sensitive, and stable. Because of their extraordinary sensitivity and stability, QCMs have been applied in recent years as biosensors for the online detection of biomolecules (Cavic et al., 1999; Marx, 2003; Nakamura and Karube, 2003). However, there are very limited applications of QCMs to the detection of heavy metal ions (Nomura and Iijima, 1981; Nomura and Sakai, 1986; Jane and Shih, 1995; Ng et al., 1998; Price et al., 2002; Casilli et al., 2004). Bipyridinium-coated electrodes detect metal ions in the millimolar range (Casilli et al., 2004). Polythiophenes-functionalized QCMs detect mercury and silver ions with a sensitivity of 10 Hz/ppm (Ng et al., 1998). Crown ether-coated electrodes combined with a conductivity measurement detect metal ions at a sensitivity of 50 Hz/ppm with a detection limit of 3×10⁻⁵ M (Jane and Shih, 1995).

Atomic adsorption spectroscopy (Hwang and Jiang, 1996), inductively coupled plasma optical emission spectroscopy (Jackson and Chen, 1996), inductively coupled plasma mass spectroscopy (Burlingame et al., 1996), high-performance liquid chromatography (Das-Ak Chakraborty et al., 1996), electrochemical separation (Hissner et al., 1996), and capillary electrophoresis (Wen and Cassidy, 1996) are commonly used techniques in metal ion detection. For real-time detection, application of amperometric method detects nanomolar to femtomolar amounts of metal ions (Ugo et al., 1998; Bontidean et al., 1998). Although femtomolar detection has been achieved with various methods, application of QCMs for the detection of heavy metal ions is still worth investigating due to its economy and convenience.

Melanin belongs to a category of phenolic polymers found in the black pigments of plants, animals, and microbes. Animal melanin is commonly divided into two types: black eumelanins and reddish-brown pheomelanins (Nicolaus, 1968; Crippa et al., 1989; Prota, 1998). The primary function of melanin is to protect the organism from the sun's direct radiation. In addition, melanin is (1) an antioxidant that possesses free radical scavenging activity, (2) a heavy metal chelator, and (3) an absorber of toxic organic species (Schwabe et al., 1989; Kollias et al., 1991; Liu et al., 1993; Smit et al., 2001; Hung et al., 2002a; Hung et al., 2002b; Sava et al., 2002; Hung et al., 2003; Sava et al., 2003; Hung et al., 2004a; Hung et al., 2004b; Izumi et al., 2005).

Recently, melanin has been extracted from Thea sinensis Linn (Sava et al., 2001a; Sava et al., 2001b). Tea melanin (TM) performs a wide range of biochemical and pharmacological roles in animals, including antioxidants (or free radical scavengers), and in particular heavy metal ion chelators (Hung et al., 2002a; Hung et al., 2002b; Sava et al., 2002; Hung et al., 2003; Sava et al., 2003; Hung et al., 2004a; Hung et al., 2004b). Applying melanin onto QCM electrodes might facilitate the development of heavy metal ion sensors (Hepel and Mahdavi, 1997; Hepel et al., 1997; Hepel, 1999; Diaz et al., 2005).

### SUMMARY OF THE INVENTION

This invention is to provide a quartz crystal microbalance (QCM) which is used to detect metal ions, and the electrode on the QCM is coated by melanin. More specifically, the melanin is tea melanin and extracted from Thea sinensis Linn.

In another aspect, the present invention provides a method of detecting metal ions in a liquid sample, comprising the following steps: (a) preparing a liquid sample containing a species of metal ions; (b) detecting various concentrations of said species of metal ions with a melanin-coated electrode to acquire a specific resonant frequency for each of the various concentrations, and inferring a standard curve from the acquired frequencies; (c) acquiring the resonant frequency of the liquid sample of step (a); and (d) comparing the specific frequency acquired in step (c) to the standard curve of step (b) to obtain the concentration of the metal ions in the liquid sample.

### BRIEF DESCRIPTION OF THE DRAWINGS

The foregoing summary, as well as the following detailed description of the invention, will be better understood when read in conjunction with the appended drawings. It should be understood, however, that the invention is not limited to the precise arrangements and instrumentalities shown.

In the drawings:

FIG. 1 is an illustration of the compositions and functions of the QCM-FIA system. (a) is a sample injection module; (b) is a sample transfer module; (c) is a detection module; (d) is a control module; (e) is an oscillation circuit; (f) is a frequency counter; and (g) is an analysis module.

FIG. 2 is the results of metal ion detection by the QCM-FIA system. Metal ions at various concentrations were injected into the QCM-FIA system, from which the corresponding frequency shifts were obtained. This plot contains all metal ions causing down-shift in resonant frequency, including Hg²⁺, Sn²⁺, Ge⁴⁺, Li⁺, Zn²⁺, Cu²⁺, Bi³⁺, Co²⁺, Al³⁺, Ni²⁺, Ag⁺, and Fe³⁺. The inset is a magnification of the 0-6 ppm region containing Hg²⁺, Sn²⁺, Ge⁴⁺, Li⁺, Zn²⁺, and Cu²⁺. Due to the ultra-high sensitivity, Hg²⁺ exhibits vertical lines even in the inset.

FIG. 3 is the results of detection of Mn⁷⁺, Pb²⁺, Cd²⁺, and Cr³⁺ by the QCM-FIA system, shown in concentration vs. frequency. Binding of these ions up-shifted QCM resonant frequency, which is atypical for QCM detection. Cd²⁺ shows a slope of 350 Hz/ppm. Others exhibit thresholds of ca. 0.4-1.8 ppm and with steeper drops in frequency shift. Relative standard deviation (R.S.D.) is shown as error bands in each curve.

### DETAILED DESCRIPTION OF THE INVENTION

This invention is to provide a modification on a quartz crystal microbalance (QCM) which is used to detect metal ions. The term “melanin-coated electrode” used herein refers to an electrode coated with melanin. According to the invention, the sensitivity of the QCM can be raised since melanin coated on the electrode can conjugate with metal ions specifically. In one embodiment of the invention, the melanin is tea melanin and extracted from Thea sinensis Linn. According to an example of the invention, the amount of tea melanin coated on the electrode is 0.1-100 ng in total. More preferably, the amount of tea melanin coated on the electrode is 1-10 ng in total. In a preferred embodiment, the amount of tea melanin coated on the electrode is 5.3 ng (i.e., 0.38 pmol) in total.

As used herein, the term “quartz crystal microbalance (QCM)” refers to a very sensitive tool to detect the change in weight. The basis of the QCM is thin quartz exhibiting the inverse piezoelectric effect. Applying an alternating current excites a mechanical oscillation of the quartz plate. Using the specific resonant frequency of the quartz leads to a direct proportionality of the mass load and the frequency change. Most metal electrodes on the quartz are made by gold, silver, aluminum or nickel. Because gold is inert, most metal electrodes are made by gold. In one embodiment, the QCM has golden electrodes of 5 mm in diameter and 100 nm in thickness.

As used herein, the term “melanin” refers to a category of phenolic polymers found in the black pigments of plants, animals, and microbes. In the present invention, melanin can be extracted from any species of theaceous plants. In one embodiment of the invention, the theaceous plant is Thea sinensis Linn. Any type of melanin may be used in the invention. In one example of the invention, melanin was extracted from Thea sinensis Linn by any standard or commonly used method by those skilled in the art. In one embodiment here, the tea melanin is purified by acid hydrolysis and centrifugation, and sequestered from any impurity to improve its homogeneity, as disclosed in U.S. Pat. No. 6,192,062. According to the invention, the sensitivity of the QCM can be raised by coating melanin of a high purity on the golden electrode.

As used herein, the term “heavy metal” refers to a metal which has density of 5 g/cm³ or more, or atomic weight of 40 or more. As shown in the following Example 4, the heavy metal ions which can be detected by the QCM include but not limited to: Hg²⁺, Sn²⁺, Ge⁴⁺, Li^{+, Zn}²⁺, Cu²⁺, Bi³⁺, Co²⁺, Al³⁺, Ni²⁺, N²⁺, Ag⁺, Fe³⁺, Mn⁷⁺, Pb²⁺, Cd²⁺ and Cr³⁺.

The term “sensitivity” used herein refers to the negative frequency shift per applied weight. The basis of a QCM is that the specific resonant frequency of the quartz leads to a direct proportionality of the mass load and the frequency change. When the shift in frequency leads to a proportionality of the smallest mass change. In other words, one ppm can induce the largest shift in frequency, the sensitivity is higher.

The term “limit of detection (LOD)” used herein refers to three times standard deviation of background noise. Numbers in parentheses are threshold concentrations for positively responding ions. The higher the sensitivity, the lower concentration can be detected. The other term “saturation point” used herein refers to the upper limit of frequency shift plot. When the saturation point is reached, all of the melanin on the golden electrode is conjugated with the metal ions.

The present invention provides a method of detecting metal ions in a liquid sample, comprising the following steps: (a) preparing a liquid sample containing a species of metal ions; (b) detecting various concentrations of said species of metal ions with a melanin-coated electrode to acquire a specific resonant frequency for each of the various concentrations, and inferring a standard curve from the acquired frequencies; (c) acquiring the resonant frequency of the liquid sample of step (a); and (d) comparing the specific frequency acquired in step (c) to the standard curve of step (b) to obtain the concentration of the metal ions in the liquid sample.

The practices and advantages of this invention are further illustrated by the following examples, which are provided for the purpose of demonstration rather than limitation.

### EXAMPLE 1

### Isolation of Team Melanin (TM)

TM was isolated using a procedure reported previously (Sava et al., 2001a; Sava et al., 2001b), except that the extraction time was reduced to 12 h to avoid excessive oxidation of the TM. After extraction, the mixture was filtered and centrifuged at 15,000×g for 30 min to obtain the TM extract. This extract was acidified to pH 2.5 through the addition of 2 N HCl and then centrifuged at 15,000×g for 15 min to form a pellet. Acid hydrolysis was employed to purify the TM. The purified product was dissolved in 0.2% NH₄OH and then precipitated. This precipitation procedure was repeated three times to sequester TM from any low-molecular-weight impurities and to improve its homogeneity. The preparation was treated with TiCl₃, as described previously (Hung et al., 2002a; Hung et al., 2002b) to fully reduce the functional groups and to improve the homogeneity of the monomers. The solutions were filtered through a Nalgene 0.45 μm syringe filter. Finally, TM was purified on a Sephadex G-75 column using 50 mM phosphate buffer (pH 7.5). The purified product was dissolved in 0.2% NH₄OH up to a concentration of 0.5%. A fraction of molecular weight 14±3 kDa was isolated after purification through a Sephadex G-75 column; its purity, as examined by HPLC, was above 95%. Further study was performed using this purified fraction. Evidence from destructive alkaline fusing, thin-layer chromatography (TLC), and mass spectrometry (MS) indicates that TM is composed mainly of flavonoids (Sava et al., 2001a; Sava et al., 2001b). The apparent molecular weight of 14 kDa is equivalent to ca. 27 theaflavins in a melanic chain.

### EXAMPLE 2

### Immobilization of Melanin on Gold Electrode

The fabricated quartz was immersed sequentially in 95% ethanol for 10 min, 1.2N NaOH for 20 min, Milli-Q water for 10 min, 1.2 M HCl for 5 min, Milli-Q water for 5 min, ethanol for 5 min, and air-dried. The gold electrode was reacted with 20 μL 2.5% glutaraldehyde at room temperature for 30 min, followed by Milli-Q water washing for 5 min. The activated electrode was reacted with 60 μL 0.5% TM for another 30 min, washed with Milli-Q water for 5 min, and allowed to air dry. A total of 5.3 ng TM (0.38 pmol) were coated onto the gold electrode.

### EXAMPLE 3

### Construction of QCM-FIA System and Preparation Before Use

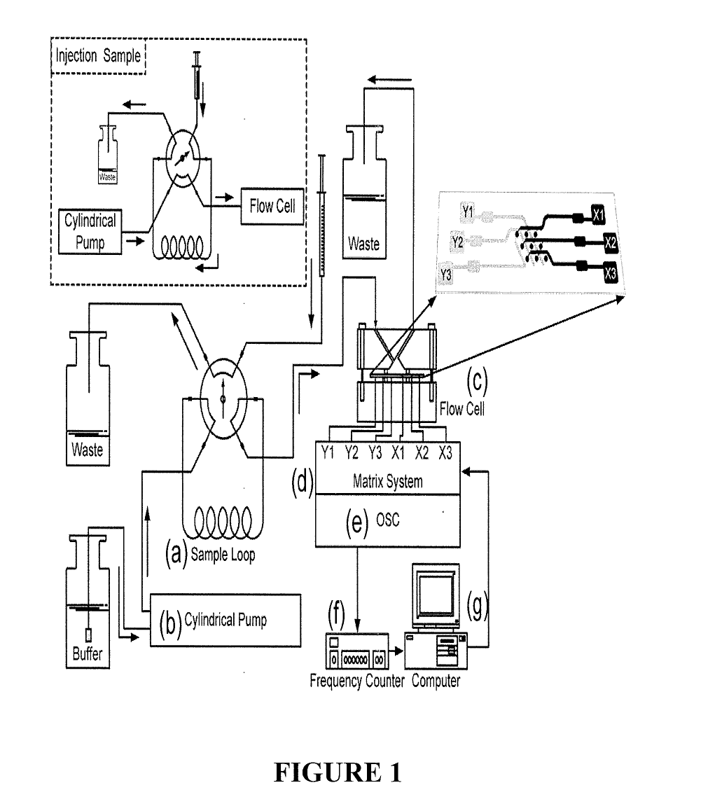

The oscillator (Catalog#35366-10) and flow-cell (Catalog#35363) were purchased from International Crystal Manufacturing Co. (Oklahoma City, USA). The QCM was fabricated from a 0.2 mm-thick AT-cut quartz wafer. The dimensions of gold electrodes were 5 mm in diameter and 100 nm in thickness. A laboratory-constructed transistor-transistor logic integrated circuit (TTL-IC) was used to power the QCM. The TTL-IC was based on IC 74HC93, 74LS138, 74LS95, and 74LS04 (Kuan Hsi Co., Taiwan). An Agilent HP 53132 Universal Frequency Counter was used to monitor the frequency output. The melanin-coated QCM was assembled into a flow-cell unit. The final FIA system consisted of a cylindrical pump, injector, flow-cell, oscillation circuit, frequency counter, and personal computer (see FIG. 1). The fundamental resonant frequency for the QCM-FIA system was 9.995 MHz. All analyses were carried out using Milli-Q ultra-pure water (18.2 MΩ cm) and solutions of analytical grade at 25° C. The QCM-FIA system was pre-run at 3 mL/h for 30 min. A volume of 5 μL was applied for each injection. The TM-coated electrode was regenerated by 50 μL injection of 0.1N HCl followed by 10 min Milli-Q water wash. Repeated regeneration caused a decrease in binding capacity but did not affect sensitivity. The TM-coated electrode was regenerated five times for optimal detection. The crystal was repeatedly coated, cleaned, and washed until the gold electrode fell off.

### EXAMPLE 4

### Detection of Metal Ions by the QCM-FIA System

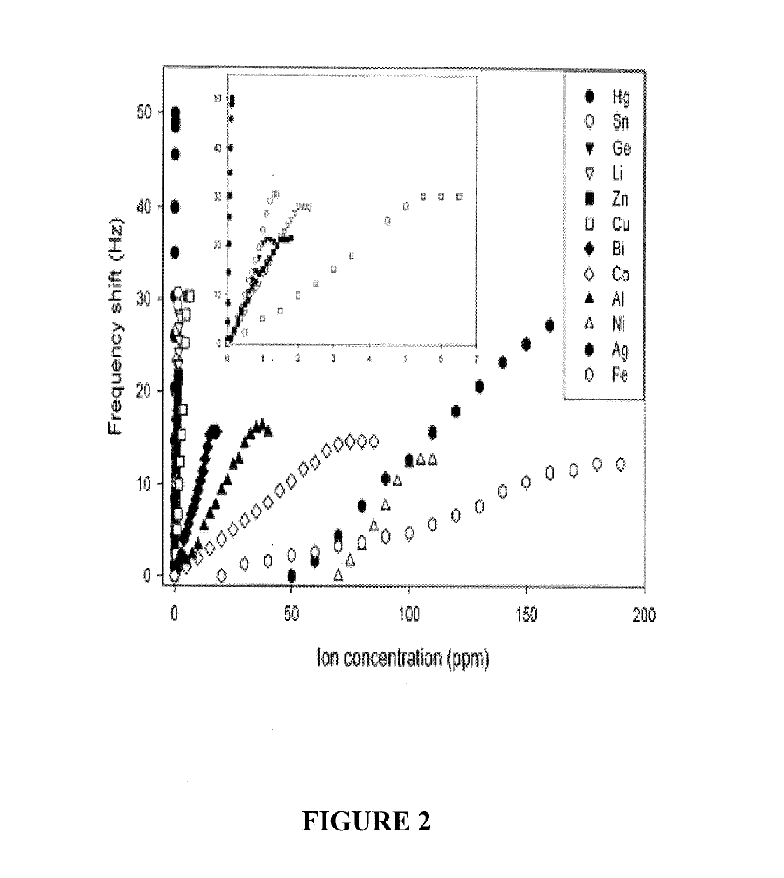

Five microliters of metal ions at various concentrations were injected into the QCM-FIA system and monitored by resonant frequency in real time. Detection of metal ions including Hg²⁺, Sn²⁺, Ge⁴⁺, Li⁺, Zn²⁺, Cu²⁺, Bi³⁺, Co²⁺, Al³⁺, Ni²⁺, Ag⁺, and Fe³⁺ was performed. Binding of metal ions onto a bare electrode and a dextran-coated electrode was performed in parallel as control experiments. For both control experiments, no frequency drop was observed up to 10 ppm Hg²⁺. Binding of metal ions onto the TM-coated electrodes decreased the resonant frequency. The negative frequency shift per applied weight, defined as sensitivity, remained constant at initial metal ion concentrations and reached a plateau at higher concentrations. The sensitivity, limit of detection (LOD), and saturation point were obtained from FIG. 2 and listed in Table 1. In particular, mercury ion exhibited distinct sensitivity of 518±37 Hz/ppm with the limit of detection (LOD) at 0.005 ppm and the saturation point at 0.55 ng. Sn²⁺, Ge⁴⁺, Li⁺, Zn²⁺, and Cu²⁺ exhibited medium-strength binding affinity with the sensitivity ranging from 5.3±1.5 Hz/ppm to 26±7 Hz/ppm while Bi³⁺, Co²⁺, Al³⁺, Ni²⁺, Ag⁺, and Fe²⁺ showed much lower sensitivity (Table 1). In summary, melanin-coated electrodes are capable of selectively binding various metal ions.

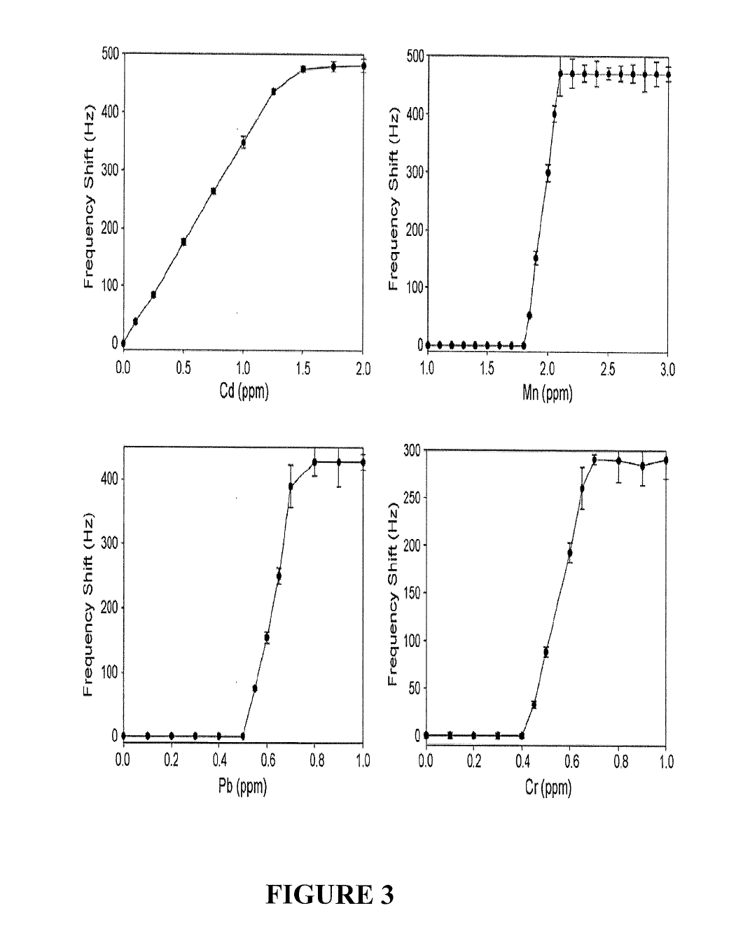

Unexpectedly, when a group of ions including Mn⁷⁺, Pb²⁺, Cd²⁺, and Cr³⁺ was injected into the QCM-FIA system, a raise in resonant frequency was observed (see FIG. 3). This group of ions showed high sensitivities ranging from 350 Hz/ppm to 2500 Hz/ppm (Table 1).

TABLE 1
Summary of binding reaction of metal ions to the tea melanin-coated
QCM

| | | | | Saturation |
|---|---|---|---|---|
| | | Sensitivity* | Limit of | point*** |
| Ion | Compound | (Hz/ppm) | detection** (ppm) | (ng) |
| Hg²⁺ | HgCl₂ | 518 ± 37 | 0.005 | 0.55 |
| Sn²⁺ | SnCl₂ | 26 ± 7 | 0.1 | 6.5 |
| Ge⁴⁺ | GeO₂ | 21.4 ± 0.1 | 0.1 | 5 |
| Li⁺ | Li₂Co₃ | 15 ± 1.2 | 0.2 | 7.5 |
| Zn²⁺ | Zn(NO₃)₂ | 14.42 ± 2.1 | 0.1 | 27.5 |
| Cu²⁺ | Cu(NO₃)₂ | 5.3 ± 1.5 | 0.5 | 75 |
| Bi³⁺ | Bi(NO₃)₃ | 1 ± 0.9 | 1.2 | 375 |
| Co²⁺ | Co(NO₃)₂ | 0.2 ± 0.2 | 5.1 | 9.5 |
| Al³⁺ | Al(NO₃)₃ | 0.52 ± 0.16 | 5.3 | 150 |
| Ni²⁺ | Ni(NO₃)₂ | 0.43 ± 0.14 | 72.5 | 525 |
| Ag⁺ | AgNO₃ | 0.23 ± 0.24 | 65.8 | 850 |
| Fe³⁺ | Fe₂O₃ | 0.05 ± 0.03 | 30.8 | 900 |
| Mn⁷⁺ | KMnO₄ | −2000 | (1.8) | — |
| Pb²⁺ | Pb(NO₃)₂ | −2500 | (0.5) | — |
| Cd²⁺ | CdCl₂ | −348.9 | 0.02 | — |
| Cr³⁺ | Cr(NO₃)₃ | −1000 | (0.4) | — |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCE

Bontidean et al., 1998 I. Bontidean, C. Berggren, G. Johansson, E. Csoregi, B. Mattiasson, J. R. Lloyd, K. J. Jakeman and N. L. Brown, Anal. Chem. 70 (19) (1998), pp. 4162-4169.
Casilli et al., 2004 S. Casilli, C. Malitesta, S. Conoci, S. Petralia, S. Sortino and L. Valli, Biosens. Bioelectron. 20 (6) (2004), pp. 1190-1195.
Cavic et al., 1999 B. A. Cavic, G. L. Hayward and M. Thompson, Analyst 124 (10) (1999), pp. 1405-1420.
Crippa et al., 1989 R. Crippa, V. Horak, G. Protta, P. Svoronos and L. Wolfram, Chemistry of Melanins. The Alkaloids vol. 36, Academic Press Inc. (1989) pp. 253-323.
Diaz et al., 2005 P. Diaz, Y. Gimeno, P. Carro, S. Gonzalez, P. L. Schilardi, G. Benitez, R. C. Salvarezza and A. H. Creus, Langmuir 21 (2005), pp. 5924-5930.
Hepel, 1999 M. Hepel In: A. Wieckowski, Editor, Interfacial Electrochemistry, Marcel Dekker, New York (1999), pp. 599-630.
Hepel and Mahdavi, 1997 M. Hepel and F. Mahdavi, Microchem. J. 56 (1997), pp. 54-64.
Hepel et al., 1997 M. Hepel, Z. Xingmin, R. Stephenson and S. Perkins, Microchem. J. 56 (1997), pp. 79-92.
Hung et al., 2002a Y. C. Hung, V. M. Sava, C. L. Juang, T. Yeh, W. C. Shen and G. S. Huang, J. Ethnopharmacol. 79 (1) (2002), pp. 75-79.
Hung et al., 2002b Y. C. Hung, V. M. Sava, S. Y. Makan, J. T.-S. Chen, M.-Y. Hong and G. S. Huang, Food Chem. 78 (2002), pp. 233-240.
Hung et al., 2003 Y. C. Hung, V. M. Sava, V. A. Blagodarsky, M. Y. Hong and G. S. Huang, Life Sci. 72 (9) (2003), pp. 1061-1071.
Hung et al., 2004a Y. C. Hung, V. Sava, M. Y. Hong and G. S. Huang, Life Sci. 74 (16) (2004), pp. 2037-2047.
Hung et al., 2004b Y. C. Hung, V. M. Sava, S. Y. Makan, M. Y. Hong and G. S. Huang, J. Agric. Food Chem. 52 (16) (2004), pp. 5284-5289.
Hwang and Jiang, 1996 T. J. Hwang and S. J. J. Jiang, Anal. Atom. Spectrosc. 11 (5) (1996), p. 353.
Izumi et al., 2005 Y. Izumi, H. Sawada, N. Yamamoto, T. Kume, H. Katsuki, S. Shimohama and A. Akaike, J. Neurosci. Res. 17 (2005), p. 17.
Jane and Shih, 1995 Y.-S. Jane and J.-S. Shih, Analyst 1 (120) (1995), pp. 517-522.
Kollias et al., 1991 N. Kollias, R. M. Sayre, L. Zeise and M. R. Chedekel, J. Photochem. Photobiol B 9 (2) (1991), pp. 135-160.
Liu et al., 1993 Y. T. Liu, M. J. Sui, D. D. Ji, I. H. Wu, C. C. Chou and C. C. Chen, J. Invertebr. Pathol. 62 (2) (1993), pp. 131-136.
Marx, 2003 K. A. Marx, Biomacromolecules 4 (5) (2003), pp. 1099-1120.
Nakamura and Karube, 2003 H. Nakamura and I. Karube, Anal. Bioanal. Chem. 377 (2003), pp. 446-468.
Ng et al., 1998 S. C. Ng, X. C. Zhou, Z. K. Chen, P. Miao, H. S. O. Chan, S. F.-Y. Li and P. Fu, Langmuir 14 (1998), pp. 1748-1752.
Nicolaus, 1968 R. Nicolaus, Melanins, Hermann, Paris (1968).
Nomura and Iijima, 1981 T. Nomura and M. Iijima, Anal. Chim. Acta 131 (1981), pp. 97-102.
Nomura and Sakai, 1986 T. Nomura and M. Sakai, Anal. Chim. Acta 183 (1986), pp. 301-305.
Price et al., 2002 G. J. Price, A. A. Clifton, V. J. Burton and T. C. Hunter, Sens. Actuators B 84 (2002), pp. 208-213.
Prota, 1998 G. Prota, Melanins and Melanogenesis, Academic Press, San Diego (1998).
Sava et al., 2001a V. Sava, B. Galkin, M.-Y. Hong, P.-C. Yang and G. S. Huang, Food Res. Int. 34 (2001), pp. 337-343.
Sava et al., 2001b V. Sava, S.-M. Yang, M.-Y. Hong, P.-C. Yang and G. S. Huang, Food Chem. 73 (2) (2001), pp. 177-184.
Sava et al., 2002 V. M. Sava, Y.-C. Hung, B. N. Golkin, M.-Y. Hong and G. S. Huang, Food Res. Int. 35 (2002), pp. 619-626.
Sava et al., 2003 V. M. Sava, Y. C. Hung, V. A. Blagodarsky, M. Y. Hong and G. S. Huang, Food Res. Int. 36 (2003), pp. 505-511.
Schwabe et al., 1989 K. Schwabe, G. Lassmann, W. Damerau and H. Naundorf, J. Cancer Res. Clin. Oncol. 115 (6) (1989), pp. 597-600.
Smit et al., 2001 N. P. Smit, A. A. Vink, R. M. Kolb, M. J. Steenwinkel, P. T. van den Berg, F. van Nieuwpoort, L. Roza and S. Pavel, Photochem. Photobiol. 74 (3) (2001), pp. 424-430.
Ugo et al., 1998 P. Ugo, L. M. Moretto, P. Bertoncello and J. Wang, Electroanalysis 10 (1998), pp. 1017-1021.

What is claimed is:

1. A quartz crystal microbalance for detecting metal ions, wherein the electrode is sensitized by melanin.

2. The quartz crystal microbalance of claim 1, wherein the melanin is tea melanin.

3. The quartz crystal microbalance of claim 2, wherein the tea melanin is prepared from Thea sinensis Linn.

4. The quartz crystal microbalance of claim 1, wherein the melanin coated on the electrode is in an amount of 0.1-100 ng in total.

5. The quartz crystal microbalance of claim 4, wherein the melanin coated on the electrode is in an amount of 1-10 ng in total.

6. The quartz crystal microbalance of claim 5, wherein the melanin coated on the electrode is in an amount of 5.3 ng in total.

7. The quartz crystal microbalance of claim 1, wherein the metal ions are heavy metal ions.

8. The quartz crystal microbalance of claim 7, wherein the heavy metal ions are selected from the group consisting of: Hg²⁺, Sn²⁺, Ge⁴⁺, Li⁺, Zn²⁺, Cu²⁺, Bi³⁺, Co²⁺, Al³⁺, Ni²⁺, Ag⁺, Fe³⁺, Mn⁷⁺, Pb²⁺, Cd²⁺, and Cr³⁺.

9. The quartz crystal microbalance of claim 2, wherein the tea melanin is obtained by purifying via acid hydrolysis and centrifugation, and sequestered from any impurity.

10. A a method of detecting metal ions in a liquid sample, comprising the following steps:
(a) preparing a liquid sample containing a species of metal ions;
(b) detecting various concentrations of said species of metal ions with a melanin-coated electrode to acquire a specific resonant frequency for each of the various concentrations, and inferring a standard curve from the acquired frequencies;
(c) acquiring the resonant frequency of the liquid sample of step (a); and
(d) comparing the specific frequency acquired in step (c) to the standard curve of step (b) to obtain the concentration of the metal ions in the liquid sample.

11. The method of claim 10, wherein the melanin is tea melanin.

12. The method of claim 11, wherein the tea melanin is prepared from Thea sinensis Linn.

13. The method of claim 10, wherein the melanin coated on the electrode is in an amount of 0.1-100 ng in total.

14. The method of claim 13, wherein the melanin coated on the electrode is in an amount of 1-10 ng in total.

15. The method of claim 14, wherein the melanin coated on the electrode is in an amount of 5.3 ng in total.

16. The method of claim 10, wherein the metal ions are heavy metal ions.

17. The method of claim 16, wherein the heavy metal ions are selected from the group consisting of: Hg²⁺, Sn²⁺, Ge⁴⁺, Li⁺, Zn²⁺, Cu²⁺, Bi³⁺, Co²⁺, Al³⁺, Ni²⁺, Ag⁺, Fe³⁺, Mn⁷⁺, Pb²⁺, Cd²⁺ and Cr³⁺.

18. The method of claim 11, wherein the tea melanin is obtained by purifying via acid hydrolysis and centrifugation, and sequestered from any impurity.
